(19) United States
(12) Patent Application Publication
(10) Pub. No.: US 2023/0348224 A1
(43) Pub. Date: Nov. 2, 2023

(54) Oxygen Tubing Reel Device

(71) Applicant: Amy Downey, Blue Springs, MO (US)

(72) Inventor: Amy Downey, Blue Springs, MO (US)

(21) Appl. No.: 18/163,928

(22) Filed: Feb. 3, 2023

Related U.S. Application Data
(60) Provisional application No. 63/324,802, filed on Mar. 29, 2022.

(51) Int. Cl. A61M 39/08; B65H 75/44

(52) U.S. Cl. CPC B65H 75/4484; A61M 39/08

(57) ABSTRACT

The present invention relates to an oxygen tubing reel device that is primarily comprised of a housing with at least one motor that rotates at least one reel that stores a tubing. The motor may be automatically engaged via a proximity sensor that extends or retracts the tubing into or out of the housing as a user moves closer or farther away from the housing. The motor may also be manipulated via a control.

### CROSS-REFERENCE TO RELATED APPLICATION

The present application claims priority to, and the benefit of, U.S. Provisional Application No. 63/324,802, which was filed on Mar. 29, 2022, and is incorporated herein by reference in its entirety.

### FIELD OF THE INVENTION

The present invention relates generally to the field of oxygen tanks and oxygen tubing. More specifically, the present invention relates to an oxygen tubing reel device that is primarily comprised of a housing with at least one motor that rotates at least one reel that stores a tubing. The motor may be automatically engaged via a proximity sensor that extends or retracts the tubing into or out of the housing as a user moves closer or farther away from the housing. The motor may also be manipulated via a control. Accordingly, the present disclosure makes specific reference thereto. Nonetheless, it is to be appreciated that aspects of the present invention are also equally applicable to other like applications, devices, and methods of manufacture.

### BACKGROUND

Many individuals are required to use an oxygen tank and related equipment. However, the attached oxygen tubing that runs from an oxygen tank may trip a user while the user is walking away or toward the tank. This may cause serious injury. Wheelchair-bound individuals who use oxygen may experience the oxygen tubing becoming caught in the wheels of the wheelchair, which is undesirable. In addition, when walking away from an oxygen tank, a user must ensure they have enough oxygen tubing to travel away from the tank. This length of tubing must then be re-wrapped when the user once again moves closer to the tank.

Therefore, there exists a long-felt need in the art for an improved device that can be used with oxygen tanks. More specifically, there exists a long-felt need in the art for an oxygen tubing reel device that prevents a user from tripping over oxygen tubing. In addition, there exists a long-felt need in the art for an oxygen tubing reel device that prevents oxygen tubing from becoming caught in a wheelchair. Further, there exists a long-felt need in the art for an oxygen tubing reel device that automatically manages the length of oxygen tubing as a user moves away from an oxygen tank.

The subject matter disclosed and claimed herein, in one embodiment thereof, comprises an oxygen tubing reel device. The device is primarily comprised of a housing with at least one motor that rotates at least one reel that stores a length of tubing. The motor may be automatically engaged via a proximity sensor that extends or retracts the tubing into or out of the housing as a user moves closer or farther away from the housing. The motor may also be manipulated via a control. The device may also be comprised of a pole that can be attached to a wheelchair, wherein the housing attaches to the pole, and a loop of the pole prevents the tubing from becoming entangled in the wheels of a wheelchair.

In this manner, the oxygen tubing reel device of the present invention accomplishes all of the foregoing objectives and provides an oxygen tubing reel device that prevents a user from tripping over oxygen tubing and prevents oxygen tubing from becoming caught in a wheelchair. Further, the device automatically manages the length of oxygen tubing as a user moves away from an oxygen tank.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subj ect matter disclosed and claimed herein, in one embodiment thereof, comprises an oxygen tubing reel device. The device is primarily comprised of a housing with at least one motor that rotates at least one reel that stores a tubing. The housing is comprised of at least one lid that attaches to the housing via at least one hinge. The lid is further comprised of at least one opening to allow a length of tubing stored on and around the reel to exit the housing. The tubing may be made from any medical tubing material known in the art but is preferably made from a 4-inch non-kink tubing. The housing can be attached to any nearby structure, object, or item via at least one strap comprised of at least one fastener that engages at least one reciprocating fastener on the top surface of the housing to form a loop.

At least one motor is positioned within the interior space and is powered by at least one battery. The motor is preferably an electric motor, wherein the reel is preferably removably attached to the motor. The rotation direction of the motor can be controlled by at least one control which may be a clicking switch, a button, or any other control that allows a user to control the motor to rotate clockwise or counterclockwise and turn off the motor. In this manner, the reel can be spun such that tubing around the reel can be extended from or retracted into the housing via the opening.

The motor may also or alternately be operated by at least one sensor, such as a proximity sensor, that automatically rotates the motor such that tubing is extended from the housing as a user moves away from the housing and oxygen tank. Conversely, the proximity sensor may automatically rotate the motor such that tubing is retracted from the housing as a user moves closer to the housing.

In one embodiment, the device is also comprised of at least one pole that is generally L-shaped and has at least one telescopic and height-adjustable vertical arm that can be secured into position via at least one fastener. One end of at least one horizontal arm is further comprised of at least one ring. The housing can be suspended from the arm via the strap and wherein the ring prevents the tubing from being caught in the wheels of the wheelchair. The vertical arm may be removably attached to the wheelchair via at least one fastener.

Accordingly, the oxygen tubing reel device of the present invention is particularly advantageous as it provides an improved device that can be used with oxygen tanks that prevents a user from tripping over oxygen tubing and prevents oxygen tubing from becoming caught in a wheelchair. In addition, the device automatically manages the length of oxygen tubing as a user moves away from an oxygen tank. In this manner, the oxygen tubing reel device overcomes the limitations of existing oxygen tanks and oxygen tubing known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of an oxygen tubing reel device of the present invention in accordance with the disclosed architecture;

FIG. 2 illustrates an exploded view of one potential embodiment of an oxygen tubing reel device of the present invention in accordance with the disclosed architecture; and

FIG. 3 illustrates a rear perspective view of one potential embodiment of an oxygen tubing reel device of the present invention in accordance with the disclosed architecture; and

FIG. 4 illustrates a perspective view of one potential embodiment of an oxygen tubing reel device of the present invention while attached to a wheelchair in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an improved device that can be used with oxygen tanks. More specifically, there exists a long-felt need in the art for an oxygen tubing reel device that prevents a user from tripping over oxygen tubing. In addition, there exists a long-felt need in the art for an oxygen tubing reel device that prevents oxygen tubing from becoming caught in a wheelchair. Further, there exists a long-felt need in the art for an oxygen tubing reel device that automatically manages the length of oxygen tubing as a user moves away from an oxygen tank.

The present invention, in one exemplary embodiment, is comprised of an oxygen tubing reel device. The device is primarily comprised of a housing with at least one motor that rotates at least one reel that stores a length of tubing. The housing is comprised of at least one lid that attaches to the housing via at least one hinge. The lid is further comprised of at least one opening to allow a tubing stored on and around the reel to exit the housing, wherein the tubing may be made from any medical tubing material known in the art. However, the tubing is preferably made from a 4-inch non-kink tubing. The housing can be attached to any nearby structure, object, or item via at least one strap comprised of at least one fastener that engages at least one reciprocating fastener on the top surface of the housing to form a loop.

At least one motor (that is preferably electric) is positioned within the interior space and is powered by at least one battery. The reel is preferably removably attached to the motor. The rotation direction of the motor can be controlled by at least one control which may be a clicking switch, a button, or any other control that allows a user to control the motor to rotate clockwise or counterclockwise and turn off the motor. In this manner, the reel can be spun such that tubing around the reel can be extended from or retracted into the housing via the opening. The motor may also or alternately, be operated by at least one sensor such as a proximity sensor that automatically rotates the motor such that tubing is extended from the housing as a user moves away from the housing and oxygen tank. Conversely, the proximity sensor may automatically rotate the motor such that tubing is retracted from the housing as a user moves closer to the housing.

In one embodiment, the device is also comprised of at least one pole that is generally L-shaped and has at least one telescopic and height-adjustable vertical arm that can be secured into position via at least one fastener. One end of at least one horizontal arm is further comprised of at least one ring. The housing can be suspended from the arm via the strap and wherein the ring prevents the tubing from being caught in the wheels of the wheelchair. The vertical arm may be removably attached to the wheelchair via at least one fastener.

Accordingly, the oxygen tubing reel device of the present invention is particularly advantageous as it provides an improved device that can be used with oxygen tanks that prevents a user from tripping over oxygen tubing and prevents oxygen tubing from becoming caught in a wheelchair. In addition, the device automatically manages the length of oxygen tubing as a user moves away from an oxygen tank. In this manner, the oxygen tubing reel device overcomes the limitations of existing oxygen tanks and oxygen tubing known in the art.

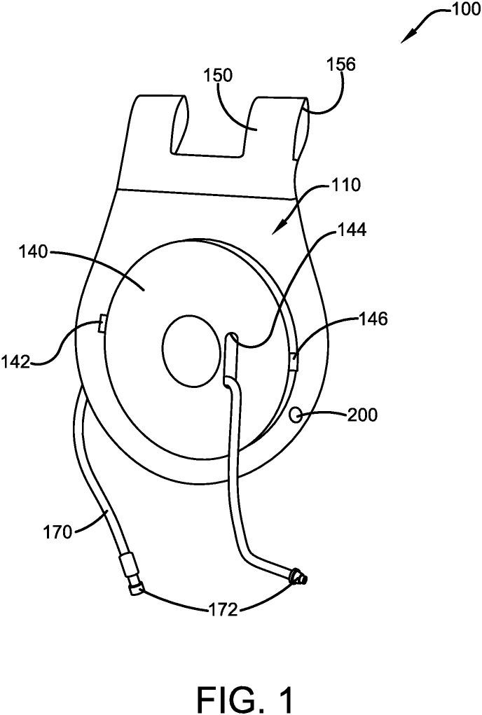

Referring initially to the drawings, FIG. 1 illustrates a front perspective view of one potential embodiment of an oxygen tubing reel device 100 of the present invention in accordance with the disclosed architecture. The device 100 is primarily comprised of a housing 110 with at least one motor 180 that rotates at least one reel 160 that stores a tubing 170. Unless otherwise specified herein, the device 100 and all components are made from a rigid or flexible plastic such as, but not limited to, acrylic, polycarbonate, polyethylene, thermoplastic, acrylonitrile butadiene styrene, low-density polyethylene, medium-density polyethylene, high-density polyethylene, polyethylene terephthalate, polyvinyl chloride, polystyrene, polylactic acid, acetal, nylon, fiberglass, recycled plastic, biodegradable plastic, etc. and/or a durable metal such as, but not limited to, stainless steel, aluminum, etc.

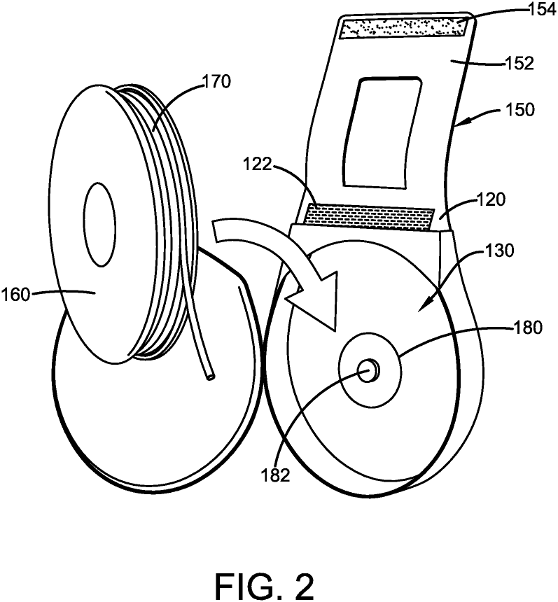

The device 100 is comprised of a housing 110 inside which at least one reel 160 is stored in an interior space 130, as best seen in FIG. 2. The housing 110 and reel 160 are preferably generally circular in shape but may be any shape known in the art. To access the interior space 130, the housing 110 is comprised of at least one lid 140 that attaches to the housing 110 via at least one hinge 142. The hinge 142 may be any hinge type known in the art such as, but not limited to, an offset blind hinge, a knuckle hinge, a butt hinge, a rising butt hinge, a gravity pivot hinge, a ball bearing hinge, a barrel hinge, a concealed hinge, a knife hinge, a piano hinge, a strap hinge, a pivot hinge, a gas-piston hinge, an injection molded hinge, a locking hinge, etc. The lid 140 is further comprised of at least one opening 144 to allow a length of tubing 190 stored on and around the reel 160 to exit the housing 110. The tubing 190 may be made from any medical tubing material known in the art but is preferably made from a 4-inch non-kink tubing. The ends 172 of the tubing 170 may have any male or female connector to attach to any oxygen tank or oxygen-related equipment known in the art. In various embodiments, the lid 140 may be the same size as the housing 110 or may only cover a portion of the housing such as, but not limited to, ⅔ of the housing to allow for ⅓ of the housing to permit the length of tubing 190 to be extended outwards from or retracted into the housing 110 such that the same does not kink. The lid 140 can be secured to the housing 110 via at least one fastener 146 such as, but not limited to, a zipper, a snap-button, a magnet, hook and loop, ties, etc.

The housing 110 can be attached to any nearby structure, object, or item via at least one strap 150. The bottom surface 152 of the strap 150 is comprised of at least one fastener 154 that engages at least one reciprocating fastener 122 on the top surface 120 of the housing 110. When connected, the fasteners 122,154 allow the strap 150 to form a generally looped shape with an opening 156. In this manner, the strap 150 can be secured around any nearby structure, object, or item. The fasteners 122,154 may be any fastener known in the art such as, but not limited to, a zipper, a snap-button, a magnet, a hook and loop fastener, a tie, adhesive, etc.

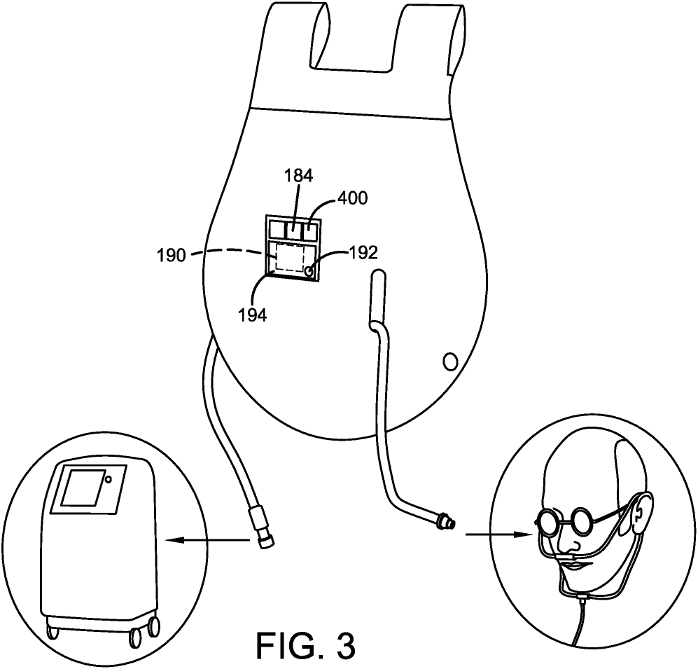

At least one motor 180 is positioned within the interior space 130 and is powered by at least one battery 190. The battery 190 may be a disposable battery 190 or a rechargeable battery 190 in the form of an alkaline, nickel-cadmium, nickel-metal hydride battery 190, etc., such as any 3 V-12 volts DC battery 190 or other conventional battery 190 such as A, AA, AAA, etc., that supplies power to the device 100. Throughout this specification the terms “battery” and “batteries” may be used interchangeably to refer to one or more wet or dry cells or batteries 190 of cells in which chemical energy is converted into electricity and used as a source of DC power. References to recharging or replacing batteries 190 may refer to recharging or replacing individual cells, individual batteries 190 of cells, or a package of multiple battery cells as is appropriate for any given battery 190 technology that may be used. The battery 190 level is indicated by at least one light 192 positioned on the housing 110, as seen in FIG. 3. The light 192 is preferably an LED light. The light 192 illuminates when the battery level is below a threshold value such as, but not limited to, 20%. In an embodiment with a removable battery 190 the battery 190 can be accessed by removing at least one battery cover 194 on the housing 110.

The motor 180 is preferably an electric motor. The reel 160 is preferably removably attached to the motor 180 via at least one fastener 182 found on the reel 160 and/or motor 180 such as, but not limited to, a bolt, a clip, a tongue and groove assembly, threads, etc. The rotation direction of the motor 180 can be controlled by at least one control 184. The control 184 may be located anywhere on the device 100 but is preferably located on the housing 110. The control 184 is preferably a clicking switch, but may also be a button or any other control that allows a user to control the motor 180, to rotate clockwise or counterclockwise and turn off the motor 180. At least one indicia 400 such as a word, symbol, or picture may instruct the user on how to operate the control 184. In this manner, the reel 160 can be spun such that tubing 170 around the reel 160 can be extended from or retracted into the housing 110 via the opening 144. In a differing embodiment, the reel 160 can be manually rotated in a clockwise or counterclockwise direction by grasping and pulling the tubing 170. In a further embodiment, only a portion of the reel 160 may rotate due to the motor 180.

The motor 180 may also or alternately, be operated by at least one sensor 200 that is in wireless electrical communication with the motor 180. In one embodiment, the sensor 200 is a proximity sensor that automatically rotates the motor 180 such that tubing 170 is extended from the housing 110 as a user moves away from the housing 110 and oxygen tank (as detected by the sensor 200). Conversely, the proximity sensor may automatically rotate the motor 180 such that tubing 170 is retracted from the housing 110 as a user moves closer to the housing 110 (as detected by the sensor 200). In a further embodiment, the sensor 200 may be a GPS sensor that may work in a similar manner. The motor 180 preferably has an automatic stop feature that stops rotation of the reel 160 to ensure all tubing 170 is not retracted into the housing 110 or all tubing 170 is not extended from the housing 110.

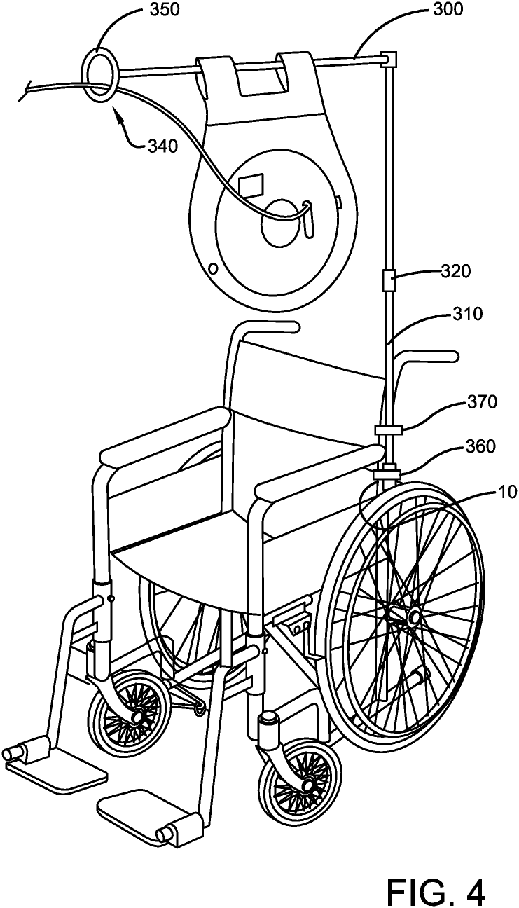

FIG. 4 illustrates a perspective view of one potential embodiment of an oxygen tubing reel device 100 of the present invention while attached to a wheelchair 10 in accordance with the disclosed architecture. In one embodiment, the device 100 is also comprised of at least one pole 300 that is generally L-shaped and has at least one telescopic, height-adjustable vertical arm 310 that can be secured into position via at least one fastener 320 such as, but not limited to, a lock. At least one horizontal arm 330 may also be length adjustable in the same manner via at least one fastener 320 such as, but not limited to, a lock. One end 340 of the horizontal arm 330 is further comprised of at least one ring 350. The housing 110 can be suspended from the arm 330 via the strap 150 and wherein the ring 350 prevents the tubing 170 from being caught in the wheels of wheelchair 10. The vertical arm 310 may be removably attached to the wheelchair via at least one fastener 360 such as, but not limited to, hook and loop strap, ties, magnets, clips, clamps, etc. In one embodiment, the stand 300 is comprised of at least one base 370. The base 370 may be any base style that allows the pole 300 to stand freely.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “oxygen tubing reel device” and “device” are interchangeable and refer to the oxygen tubing reel device 100 of the present invention.

Notwithstanding the foregoing, the oxygen tubing reel device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the oxygen tubing reel device 100 as shown in the FIGS. are for illustrative purposes only, and that many other sizes and shapes of the oxygen tubing reel device 100 are well within the scope of the present disclosure. Although the dimensions of the oxygen tubing reel device 100 are important design parameters for user convenience, the oxygen tubing reel device 100 may be of any size, shape and/or configuration that ensures optimal performance during use and/or that suits the user’s needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An oxygen tubing reel device comprising:
a housing;
a motor;
a battery;
a sensor;
a control;
a reel;
a tubing; and
a strap.

2. The oxygen tubing reel device of claim 1, wherein the reel rotates clockwise and counterclockwise relative to the motor.

3. The oxygen tubing reel device of claim 1, wherein the tubing is stored around the reel.

4. The oxygen tubing reel device of claim 1, wherein the sensor is in wireless electrical communication with the motor.

5. The oxygen tubing reel device of claim 4, wherein the sensor controls and determines a rotation direction of the motor.

6. An oxygen tubing reel device comprising:
a housing comprised of an interior space;
a motor positioned within the interior space that is configured to rotate in a clockwise direction and a counterclockwise direction;
a battery;
a sensor;
a control that controls the rotation of the motor;
a reel attached to the motor;
a tubing stored around the reel; and
a strap positioned on the housing.

7. The oxygen tubing reel device of claim 6, wherein the sensor is a proximity sensor.

8. The oxygen tubing reel device of claim 6, wherein the strap is comprised of an opening.

9. The oxygen tubing reel device of claim 6, wherein the reel is removably attached to the motor.

10. The oxygen tubing reel device of claim 6, wherein the tubing is a non-kink tubing.

11. The oxygen tubing reel device of claim 6, wherein the housing comprises a lid.

12. The oxygen tubing reel device of claim 11, wherein the lid is comprised of a fastener.

13. The oxygen tubing reel device of claim 6, wherein an end of the tubing has a male or a female connector.

14. An oxygen tubing reel device comprising:
a housing comprised of an interior space;
a motor positioned within the interior space that is configured to rotate in a clockwise direction or a counterclockwise direction;
a battery;
a sensor;
a control that controls the direction of the rotation of the motor;
a reel attached to the motor;
a tubing stored around the reel;
a strap positioned on the housing; and
a pole comprised of a vertical arm and a horizontal arm.

15. The oxygen tubing reel device of claim 14, wherein an end of the horizontal arm is comprised of a ring.

16. The oxygen tubing reel device of claim 14, wherein the vertical arm is a height-adjustable vertical arm.

17. The oxygen tubing reel device of claim 14, wherein the horizontal arm is a length-adjustable horizontal arm.

18. The oxygen tubing reel device of claim 14, wherein the pole is comprised of a fastener.

19. The oxygen tubing reel device of claim 14, wherein the housing is comprised of a first fastener and the strap is comprised of a second fastener.

20. The oxygen tubing reel device of claim 14 comprised of a light that indicates a battery level of the battery.
